(19) United States
(12) Patent Application Publication
(10) Pub. No.: US 2009/0191253 A2
(43) Pub. Date: Jul. 30, 2009

(54) Use of K-252a and Kinase Inhibitors for the Prevention or Treatment of HMGB1-Associated Pathologies

(75) Inventors: Silvano FUMERO, Ivrea (TO) (IT); Francesco PILATO, Milano (IT); Domenico BARONE, Torino (IT); Luisa BERTARIONE RAVA ROSSA, Pavone Canavese (TO) (IT); Valentina MAINERO, Ivrea (TO) (IT); Silvio TRAVERSA, Palazzo Canavese (IT)

Correspondence Address: ROTHWELL, FIGG, ERNST & MANBECK, P.C., 1425 K STREET, N.W., SUITE 800, WASHINGTON, DC, 20005, UNITED STATES, 2027836040, 2027836031, PTO-PAT-Email@rfem.com

(73) Assignees: Creabilis Therapeutics S.P.A., Colleretto Giacoso (IT); Bio3Research srl, Milano (IT)

(21) Appl. No.: 11/658,701

(22) Filed: Jan. 29, 2007

Related U.S. Application Data
(60) Provisional application No. 60/647,007, filed on Jan. 27, 2005.

(51) Int. Cl. A61F 2/04; A61K 31/407; A61P 37/02; A61P 9/00; A61K 39/395; A61K 31/7105; A61K 38/02; A61K 31/713

(52) U.S. Cl. 424/423; 514/410; 424/173.1; 424/153.1; 514/2

(57) ABSTRACT

The present invention relates to the use of K-252a, a physiologically active substance produced by microorganisms, and of its salts or synthetic and/or chemically modified derivatives for the prevention or treatment of HMGB1 associated pathologies. More particularly, the present invention relates to the use of K-252a for the prevention or treatment of restenosis.

The present invention relates to the use of K-252a, a physiologically active substance produced by microorganisms, or/and a kinase inhibitor and of its salts or synthetic and/or chemically modified derivatives for the prevention or treatment of HMGB1-associated pathologies. More particularly, the present invention relates to the use of K-252a or/and a kinase inhibitor for the prevention or treatment of restenosis.

Recent researches in the field of sepsis and inflammation have led to an improved understanding of the pathogenic mechanisms and events underlying their clinical onset and development. In the early stages of sepsis, for instance, bacterial endotoxins stimulate cells of the innate immune system which release pro-inflammatory cytokines (TNF, IL-1α and IL-6). These early cytokines, in turn, induce the release of a later-acting downstream mediator—identified as the known protein HMGB1—that triggers the pathological sequelae mediated by the subsequent release of cytokines like TNF, IL-1α, IL-1β, IL-IRa, IL-6, IL-8, etc., leading to a multisystem pathogenesis or to a lethal systemic inflammation. The HMGB1 protein belongs to the family of high mobility group (HMG) proteins. HMG proteins, so called due to their high electrophoretic mobility in polyacrylamide gels, are the most ubiquitous non-histone proteins associated with isolated chromatin in eukaryotic cells. These proteins play a generalized . . . architectural” role in DNA bending, looping, folding and wrapping since they either distort, bend or modify DNA structures complex with transcription factors or histones. The high mobility group 1 (HMGB1) protein is usually a nuclear factor, in particular a transcriptional regulatory molecule causing DNA bending and facilitating the binding of several transcriptional complexes.

Extracellularly released HMGB1 acts as a potent cytokine and as an extremely potent macrophage-stimulating factor. HMGB1 acts directly by binding to the cell membrane inducing signaling and chemotaxis, having a chemokine-like functions, and further acting indirectly by up-regulating the expression and secretion of pro-inflammatory cytokines. This makes extracellular HMGB1 protein a potent chemotactic and immunoregulatory protein which promotes an effective inflammatory immune response. Furthermore, other proteins belonging to the family of HMG-proteins and able to bend DNA are released together with HMGB1 in the extracellular medium. These proteins are inter alia HMGB2, HMGB3, HMG-1L10, HMG-4L and SP100-HMG. They share with HMGB1 highly homologous amino acid sequences. Like HMGB1, they trigger/sustain inflammatory pathologies interacting with the same receptors and leading to the same downstream pathways of interaction.

The release of HMGB1 by injured and necrotic cells has been demonstrated to actively mobilize rat smooth muscle cells (RSMC) in vitro (1) and to trigger inflammation in vivo (2).

In healthy cells, HMGB1 migrates to the cytoplasm both by passive and active transport. However, all cultured cells and resting monocytes contain the vast majority of HMGB 1 in the nucleus, indicating that in baseline conditions import is much more effective than export. Cells might transport HMGB 1 from the nucleus by acetylating lysine residues which are abundant in HMGB1, thereby neutralizing their basic charge and rendering them unable to function as nuclear localization signals. Nuclear HMGB1 hyperacetylation determines the relocation of this protein from the nucleus to the cytoplasm (in the fibroblasts, for example) or its accumulation into secretory endolysosomes (in activated monocytes and macrophages, for example) and subsequent redirection towards release through a non-classical vesicle-mediated secretory pathway. HMGB1 secretion by already activated monocytes is then triggered by bioactive lysophosphatidylcholine (LPC), which is generated later in the inflammation site from phosphatidylcholine through the action of the secretory phospholipase sPLA2, produced by monocytes several hours after activation. Therefore, secretion of HMGB1 seems to be induced by two signals (Bonaldi et al., 2003) and to take place through three steps: 1) at first, an inflammatory signal promotes HMGB1 acetylation and its relocation from the nucleus to the cytoplasm (step 1) and storage into cytoplasmic secretory vesicles (step 2); then, a secretion signal (extracellular ATP or lysophosphatidylcholine) promotes exocytosis (third step) (Andersson et al., 2002; Scaffidi et al. 2002; Bonaldi et al., 2003; Friedman et al., 2003; Gardella et al., 2002).

Released HMGB1 has been identified as one of the ligands binding to the RAGE receptor. This receptor is expressed in most cell types, and at a high level mainly in endothelial cells, in vascular smooth muscle cells, in monocytes and macrophages and in mononuclear phagocytes. Recognition involves the C-terminal of HMGB1. The interaction of HMGB1 and RAGE triggers a sustained period of cellular activation mediated by RAGE up-regulation and receptor-dependent signaling. In particular, the interaction of HMGB1 and RAGE activates several intracellular signal transduction pathways, including mitogen-activated protein kinases (MAPKs), Cdc-42, p21 ras, Rac and the nuclear translocation factor KB (NF-KB), the transcription factor classically linked to inflammatory processes (Schmidt et al., 2001).

According to several experimental evidences, released HMGB1 may also interact with the receptors belonging to the family of the Toll-like receptors (TLR), e.g. with the subclasses TLR2, TLR4, TLR7, TLR8 or/and TLR9. Furthermore, HMGB1 may also interact with the functional N-terminal lectin-like domain (D1) of thrombomodulin. Due to the ability of the functional D1 domain of thrombomodulin to intercept and bind circulating HMGB1, the interaction of the HMGB1 with the RAGE-receptors and the Toll-like receptors is prevented.

Structurally, the HMGB1 protein is a ca. 25 kDa protein with a highly conserved sequence among mammals, whereby 2 out of 214 amino acids have conservative substitutions in all mammalian species. HMGB1 is ubiquitously present in all vertebrate nuclei and, in particular, can be found in fibroblasts, neurons, hepatocytes, glia and in cells derived from hematopoietic stem cells, including monocytes/macrophages, neutrophils and platelets. The HMGB1 molecule has a tripartite structure composed of three distinct domains: two DNA binding domains called HMG Box A and Box B, and an acid carboxyl terminus, making it bipolarly charged. The two basic DNA-binding domains, called box-A and box-B, are able to recognize and bind DNA with high affinity and interact with several transcription factors and nuclear steroid receptors. They play a crucial role not only in transcription processes, but also in apoptosis (programmed cell death) induction (3-5).

Recently, it was shown that, unlike injured or necrotic cells which release HMGB1 by simple diffusion and thereby trigger inflammation, apoptotic cells avidly retain HMGB1 bound to chromatin remnants even after their eventual lysis. It has also been argued that extracellular HMGB1 is primarily a signal of tissue damage and monocytes and macrophages have “learned” to mimic an ancient alarm signal. Besides, very recently HMGB1 was shown to induce migration and proliferation of both adult and embryonic mesoanglioblasts and smooth muscle cells (2 and WO 02/074337).

K-252a (molecular weight 467.5) is a glycosylated indole carbazole isolated for the first time in 1986 (6) from Nocardiopsis sp. (U.S. Pat. No. 4,555,402 and WO 97/38120-EP 0 834 574 B1). Since it is a lipophilic molecule it is capable of crossing the membranes of living cells. K-252a is a non-specific inhibitor of the broad family of serine/threonine protein kinases such as pkA, pkC, pkG, myosin light chain kinase (7), CaM kinase II and is characterized by a nM affinity for NGF receptors (TrkA). The chemical structure of K-252a is depicted in Formula (I):

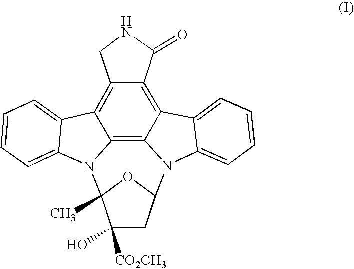

K-252a has anti-histamine releasing, anti-allergic effects (U.S. Pat. No. 4,533,402) and o an antiproliferative effect on human prostatic carcinoma cell lines (8) and on human psoriatic keratinocytes (PCT/EP03/08077). The latter activity is due to TrkA-phosphorylation blockade and consequent NGF activity inhibition. Further, it has been shown that both human and rat smooth muscle cells express NGF and its receptor TrkA (9).

Surgical procedures during angioplasty frequently induce intima injury causing the damage and necrosis of a variety of cell types, including endothelial cells. This may result in restenosis, a condition characterised by reclosure of arteries—caused by re-proliferation and re-migration of blood o vessel cells. Today, restenosis occurs in more than 20% of patients after surgical angioplasty and this condition requires a second surgery. Thus, there is a need for novel medicaments which are suitable for the prevention or treatment of restenosis.

In the present invention it has been demonstrated that K-252a (i) has a potent biological effect on HMGB1-induced smooth muscle cells migration and proliferation in response to the mechanical injury induced by surgical stent application and (ii) acts as an antagonist/inhibitor of the broad spectrum of pathological activities triggered and sustained/amplified by o HMGB1 itself in its role of pro-inflammatory chemotactic chemokine and/or by the cascade of inflammatory cytokines induced by its release.

Further, it was surprisingly found that HMGB1 is secreted by smooth muscle cells in human atherosclerotic plaques (M. Bianchi, unpublished results).

Thus, a first aspect of the present invention relates to the use of

(i) K-252a or/and

(ii) at least one kinase inhibitor,

or/and a salt or a derivative of (i) or/and (ii) for the preparation of a medicament for the prevention or treatment of HMGB1-associated pathologies. In this aspect,

(i) K-252a or/and

(ii) at least one kinase inhibitor, or/and a salt or a derivative of (i) or/and (ii) is administered in a therapeutically effective dose to a subject in need thereof in order to prevent or treat HMGB1-associated pathologies.

In the context of the present invention, HMGB1-associated pathologies include pathologies associated with the non-acetylated or/and acetylated form of HMGB1. In the context of the present invention, HMGB1-associated pathologies include further pathologies which are associated with the non-acetylated or/and acetylated form of HMGB1 homologous proteins. Preferred HMGB1 homologous proteins are HMGB2, HMGB3, HMG-1L10, HMG-4L or/and SP100-HMG. Therefore, in the use of the present invention, the HMGB1-associated pathologies are pathologies associated with the non-acetylated or/and with the acetylated form of HMGB1 or of HMGB1 homologous proteins. In the method of the present invention for the prevention or treatment of HMGB1-associated pathologies, HMGB1-associated pathologies are preferably pathologies associated with the non-acetylated or/and acetylated form of HMGB1 or of HMGB1 homologous proteins.

In the context of the present invention, “HMGBI” includes the non-acetylated form or/and the acetylated form of HMGB1. Likewise “HMGB1 homologous proteins” include the non-acetylated form or/and the acetylated form of HMGB1 homologous proteins. Preferred HMGB1 homologous proteins are HMGB2, HMGB3, HMG-1L10, HMG-4L or/and SP100-HMG.

A homologous protein of HMGB1 is defined in the context of the present invention as a protein having an amino acid sequence which has an identity on the amino acid level of at least 60%, preferably of at least 70%, more preferably of at least 80% and even more preferably of at least 90% compared to the amino acid sequence of the HMGB1 protein.

The term “identity” is understood within the context of the present invention as a percentage value which results when one divides the number of identical amino acids of two amino acid sequences which are to be compared by the number of all the amino acids of one of the two sequences.

The K-252a used in the context of the present invention can be obtained either by (i) extraction and purification from microorganism cells containing K-252a and which are obtained by culturing microorganisms capable of producing K-252a or/and by (ii) chemical synthesis (Wood et al., J., Am. Chem. Soc. 117:10413-10414, 1995). Microorganisms which produce K-252a and from which K-252a can be isolated belong preferably to the genus Nocardiopsis sp. and Saccaromyces sp.

Without wishing to be bound by theory, the circular dichroism data of the present invention and fluorescence data indicate that the inhibitory action of K-252a upon the HMGB1 activity does not necessarily depend on a direct interaction between K-252a and HMGB1. Although the mechanism by which K-252a inhibits HMGB1 activities has not yet been fully elucidated, it is more than likely that K-252a inhibits the activity of HMGB1 by inhibition of at least one kinase, such as a tyrosine kinase, a phosphokinase or/and a further kinase. Extracellular HMGB1 interacts with its membrane receptors, in particular RAGE and TLR receptors, triggering the initiation of a kinase cascade inside the cell. This cascade transports the information of the extracellular HMGB1 binding throughout the cytoplasm and in the nucleus, causing the cell to respond to the external stimulus. It is supposed that K-252a can block the cascade triggered by HMGB1 binding at different stages, and thus resulting in an overall inhibition of HMGB1 action on the cell.

The inventors of the present invention have tested the inhibition activity of K-252a on 67 human kinases and found that K-252a is capable of inhibiting several of these molecules. Indeed, 17 out of the 67 kinases tested showed an inhibition greater than 90% (cf. Example 5). Thus, K-252a has been identified as an agent for treating disorders associated with one or several kinases as shown in Table 1 and 2, for which an inhibition of at least 80 percent, preferably of at least 90 percent and more preferably of at least 95 percent has been found.

A disorder associated with a kinase is preferably a disorder associated with increased kinase activity in a diseased cell or organism compared to a non-diseased cell or organism. Kinase activity may be determined on transcript level (e.g. by measuring mRNA) or on protein level (e.g. by measuring amount and/or activity of protein).

Further, as indicated above, K-252a may be used with at least one further kinase inhibitor, an inhibitor selected from tyrosine kinase inhibitors or/and phosphokinase inhibitors. A preferred tyrosine kinase inhibitor is an inhibitor of TrkA, TrkB, TrkC or/and an inhibitor of the subfamily of tyrosine kinase receptors including the Ron receptor, c-Met (receptor of HGF/scatter factor) and Sea receptors. A preferred phosphokinase inhibitor is an inhibitor of PKA, PKC, or/and PKG. Another preferred kinase inhibitor is an inhibitor of other kinases such as Raf kinase, Ras kinase, CaM kinase, MLC kinase, MAP kinase, MEK, ERK, JUN kinase or/and PI3Kγ.

There are kinase inhibitors known in the state of the art suitable for the use of the present invention. Therefore, the at least one kinase inhibitor suitable in the present invention is preferably a known kinase inhibitor. Suitable known inhibitors of CaM kinase are calmodulin binding domain, Ca27calmodulin kinase II inhibitor 281-309, hypericin. Suitable known inhibitors of TrkA, TrkB or/and TrkC are CP 701, genistein, herbimycin, lavendustin, quercetin, radicicol. Suitable known inhibitors of MAP kinase are hymenialdisine, CP-1347, olomoucine, CC-401. Suitable known MLC kinase inhibitors are piceatannol, staurosporine, myosin light chain inhibitor peptide 18. Suitable known phosphatidylinositol-3 kinase (PI3Kγ) inhibitors are quercetin, wortmannin. Suitable known PKA inhibitors are staurosporine, KT-5720. Suitable known PKC inhibitors are staurosporine, bisindolylmaleimide 1, calphostin C, and chelerytrine. Suitable known PKG inhibitors are staurosporine, H-7, H-9, and KT-5823.

In the present invention, K-252a or/and the at least one kinase inhibitor may be employed in the form of a salt or/and derivative.

Preferred K-252a salts or/and salts of kinase inhibitors are salts with pharmaceutically acceptable cations, e.g. alkaline or alkaline-earth cations or anions, e.g. inorganic anions or organic anions. Preferred K-252a derivatives include synthetic and/or chemically modified compounds, e.g. compounds having substituents on the ring system, e.g. Ci-C4 alkyl groups, compounds wherein the methyl ester group has been replaced by another ester group, an amide group or by H or a cation, and/or compounds wherein the N-atom in the cyclic amide group is substituted with a Ci-C4 alkyl group.

An HMGB1-associated pathology is a condition in a patient wherein an increased concentration of the HMGB1 protein and/or of HMGB1 homologous proteins in the acetylated or non-acetylated form is present in the biological fluids and tissues, compared to the concentration in normal subjects where these HMGB1 proteins are practically undetectable. The HMGB1-associated pathologies and/or the pathologies associated with HMGB1 homologous proteins are pathologies with a strong inflammatory basis or pathologies which result from the stimulation of cytokine such as TNF-alpha, IL-1, IL-6 etc., or pathologies which result from toxic events, such as intoxication, infection, burn, etc. In particular high concentrations of the HMGB1 protein and homologous proteins have been found and determined in plasma of patients with sepsis, in plasma and synovial fluid of rheumatoid arthritis patients, in brains of Alzheimer's disease patients, in plasma and tissues of melanoma patients, in plasma of systemic lupus erythematosus patients, in atherosclerotic plaques of atherosclerotic patients, etc. The determination and evidence of HMGB1 protein and/or homologous proteins in biological fluids and tissues, may be detected by common diagnostic tools known by the skilled person in the art, including for example detection by ELISA assays etc.

HMGB1-associated pathologies according to the present invention are preferably pathological conditions mediated by activation of the inflammatory cytokine cascade. Non limiting examples of conditions which can be usefully treated using the present invention include the broad spectrum of pathological conditions induced by the HMGB1-chemokine and by the HMGB1-induced cascade of inflammatory cytokines grouped in the following categories: restenosis and other cardiovascular diseases, reperfusion injury, inflammation diseases such as inflammatory bowel disease, systemic inflammation response syndrome, e.g. sepsis, adult respiratory distress syndrome, etc, autoimmune diseases such as rheumatoid arthritis and osteoarthritis, obstetric and gynecological diseases, infectious diseases, atopic diseases, such as asthma, eczema, etc, tumor pathologies, e.g. solid or non-solid tumor diseases associated with organ or tissue transplants, such as reperfusion injuries after organ transplantation, organ rejection and graft-versus-host disease, congenital diseases, dermatological diseases such as psoriasis or alopecia, neurological diseases, opthalmological diseases, renal, metabolic or idiopathic diseases and intoxication conditions, e.g. iatrogenic toxicity, wherein the above diseases are caused by, associated with and/or accompanied by HMGB1 protein release.

In particular, the pathologies belonging to inflammatory and autoimmune diseases include rheumatoid arthritis/seronegative arthropathies, osteoarthritis, inflammatory bowel disease, Crohn's disease, systemic lupus erythematosus, iridoeyelitis/uveitis, optic neuritis, idiopathic pulmonary fibrosis, systemic vasculitis/Wegener's granulomatosis, sarcoidosis, orchitis/vasectomy reversal procedures. Systematic inflammatory response includes sepsis syndrome (including gram positive sepsis, gram negative sepsis, culture negative sepsis, fungal sepsis, neutropenic fever, urosepsis, septic conjunctivitis), meningococcemia, trauma hemorrhage, hums, ionizing radiation exposure, acute and chronic pancreatitis, adult respiratory distress syndrome (ARDS), prostatitis. Reperfusion injury includes post-pump syndrome and ischemia-reperfusion injury. Cardiovascular disease includes atherosclerosis, intestinal infarction, cardiac stun syndrome, myocardial infarction, congestive heart failure and restenosis. Obstetric and gynecologic diseases include premature labour, endometriosis, miscarriage and infertility. Infectious diseases include HIV infection/HIV neuropathy, septic meningitis, hepatitis B and C virus infection, herpes virus infection, septic arthritis, peritonitis, pneumonia epiglottitis, E. coli 0157:H7, haemolytic uremic syndrome/thrombolytic thrombocytopenic purpura, malaria, Dengue hemorrhagic fever, leishmaniasis, leprosy, toxic shock syndrome, streptococcal myositis, gas gangrene, mycobacterium tuberculosis, mycobacterium avium intracellulare, Pneumocystis carinii pneumonia, pelvic inflammatory disease, orchitis/epidydimitis, legionella, Lyme disease, influenza A, Epstein-Barr Virus, Cytomegalovirus, viral associated hemiaphagocytic syndrome, viral encephalitis/aseptic meningitis. Atopic disease include asthma, allergic rhinitis, eczema, allergic contact dermatitis, allergic conjunctivitis, hypersensitivity pneumonitis. Malignancies (solid and liquid tumor pathologies) include melanoma, ALL, AML, CML, CLL, Hodgkin's disease, non Hodgkin's lymphoma, Kaposi's sarcoma, colorectal carcinoma, nasopharyngeal carcinoma, malignant histiocytosis and paraneoplastic syndrome/hypercalcemia of malignancy. Transplant diseases include organ transplant rejection and graft-versus-host disease. Congenital disease includes cystic fibrosis, familial hematophagocytic lymphohistiocytosis and sickle cell anemia. Dermatologic disease includes psoriasis, psoriatic arthritis and alopecia. Neurologic disease includes neurodegenerative diseases, Alzheimer's Disease, Parkinson's Disease, multiple sclerosis, amyotrophic lateral sclerosis, migraine headache, amyloid-associated pathologies, prion diseases/Creutzfeld-Jacob disease, cerebral infarction and peripheral neuropathies. Renal disease includes nephrotic syndrome, hemodialysis and uremia. latrogenic intoxication condition includes OKT3 therapy, Anti-CD3 therapy, Cytokine therapy, Chemotherapy, Radiation therapy and chronic salicylate intoxication. Metabolic and idiopathic disease includes Wilson's disease, hemochromatosis, alpha-1 antitrypsin deficiency, diabetes, Hashimoto's thyroiditis, osteoporosis, hypothalamic-pituitary-adrenal axis evaluation and primary biliary cirrhosis. Opthalmological disease includes glaucoma, retinopathies and dry eye.

Moreover, pathologies which can be usefully treated using the present invention further include multiple organ dysfunction syndrome, muscular dystrophy, septic meningitis, iatrogenic peripheral nerve lesions, atherosclerosis, appendicitis, peptic or gastric or duodenal ulcers, ulcerative pseudomembranous, acute or ischemic colitis, diverticulitis, epiglottitis, fever, peritonitis, achalasia, cholangitis, cholecystitis, enteritis, Whipple's disease, asthma, allergic rhinitis, anaphylactic shock, immune complex disease, organ necrosis, hay fever, septicaemia, endotoxic shock, hyperpyrexia, eosinophilic granuloma, granulomatosis, sarcoidosis, septic abortion, vaginitis, prostatitis, urethritis, emphysema, rhinitis, alvealitis, bronchiolitis, pharyngitis, pneumoultramicroscopicsilico-volcanoconiosis, pleurisy, sinusitis, influenza, respiratory syncytial virus infection, disseminated bacteremia, candidiasis, filariasis, amebiasis, hydatid cyst, dermatomyositis, burns, sunburn, urticaria, warts, wheal, vasulitis, angiitis, endocarditis, pericarditis, myocarditis, arteritis, thrombophlebitis, periarteritis nodosa, rheumatic fever, celiac disease, encephalitis, cerebral embolism, Guillaume-Barre syndrome, neuritis, neuralgia, spinal cord injury, paralysis, obesity, weight loss, anorexia nervosa, cachexia, epithelial barrier dysfunction, uveitis, arthriditis, arthralgias, osteomyelitis, fasciitis, Paget's disease, gout, periodontal disease, synovitis, myasthenia gravis, Goodpasture's syndrome, Babcets's syndrome, ankylosing spondylitis, Barger's disease, Retier's syndrome, bullous dermatitis (bullous pemphigoid), alopecia pemphigous and pemphigous vulgaris, acne, benign prostatic hypertrophy, breast cancer, cervical cancer, chlamydia, cirrhosis, chronic obstructive pulmonary disease, cystitis, diarrhoea, genital herpes, genital warts, legionnaire disease, ovarian cancer, skin cancer, testicular cancer, West Nile virus infection, whooping cough.

In the context of the present invention, in the use of K-252a for the preparation of a medicament for the prevention or treatment of HMGB1-associated pathologies, pathologies such as allergies, allergy-related disorders and prostatic carcinoma are not encompassed by the scope of the present invention.

In an especially preferred embodiment, K-252a or/and the at least one kinase inhibitor is used for the prevention or treatment of cardiovascular diseases, particularly artherosclerosis and/or restenosis occurring during or after angioplasty. More preferably, the medicament is used for blocking, retarding and/or impairing connective tissue regeneration in restenosis during or after angioplasty.

By inhibiting HMGB1 activity, the migration and proliferation of smooth muscle cells (SMC) that occur during restenosis can be prevented and/or inhibited. SMCs are located in the tunica media where they are embedded in an extracellular matrix. In intact vessels, SMC cells are in contractile state and show a phenotype characterised by absence of cell division and migration which is responsible for vessel wall rigidity, elasticity maintenance and peripheral blood pressure control.

When the vessel endothelium is damaged, either after mechanical (scraped by stent insertion) or local inflammatory injuries, SMCs switch to a synthetic phenotype and undergo cell division and migration. The migration of SMCs from the tunica media to the tunica intima, resulting in intimal thickening, plays an important role in the pathophysiology of many vascular disorders, such as atherosclerosis and restenosis after coronary angioplasty. In the synthetic state, SMCs also produce higher amounts of extracellular proteinases, growth factors, cytokines and secrete a fibrous extracellular matrix. After vessel wall traumatic injury, the release of several growth factors and/or chemoattractants and cell proliferation inducers (HMGB1 and HMGB1 homologous proteins are one of the most relevant), either by activated circulating monocytes, macrophages and platelets, or by damaged endothelial cells, can induce the switch of SMC cells from the contractile to the synthetic phenotype and direct their migration towards the vessel intima. In the context of the present invention it was surprisingly found that HMGB1 and HMGB1 homologous proteins are secreted by smooth muscle cells in human atherosclerotic plaques. Thus, K252a or/and the at least one kinase inhibitor or/and derivatives thereof are suitable therapeutic agents in the prevention and/or treatment of restenosis, e.g. through systemic administration or/and through drug-eluting stents.

K-252a or/and the at least one kinase inhibitor or/and derivatives thereof may be used either alone or in combination with one or several further agents. In particular, K-252a or/and the at least one kinase inhibitor or/and derivatives thereof may be used in combination with at least one further agent capable of inhibiting an early mediator of the inflammatory cytokine cascade. For example, K-252a or/and the at least one kinase inhibitor or/and derivatives thereof may be administered together with an agent capable of inhibiting early mediators of the inflammatory cytokine cascade, e.g. an antagonist or inhibitor of a cytokine selected from the group consisting of TNF, IL-Ia1 IL-1β, IL-R3, IL-8, MIP-Ia, MIP1β, MIP-2. MIF and IL-6.

The further agent used in combination with K-252a or/and at least one kinase inhibitor or/and derivatives thereof may also be an inhibitor of RAGE, e.g. an antibody directed to RAGE, a nucleic acid or nucleic acid analogue capable of inhibiting RAGE expression, e.g. an antisense molecule, a ribozyme or a RNA interference molecule, or a small synthetic molecule antagonist of the interaction of HMGB1 with RAGE, preferably of the interaction of the non-acetylated or/and acetylated form of HMGB1 with RAGE, or soluble RAGE (sRAGE). The antibody to RAGE is preferably a monoclonal antibody, more preferably a chimeric or humanised antibody or a recombinant antibody, such as a single chain antibody or an antigen-binding fragment of such an antibody. The soluble RAGE analog may be optionally present as a fusion protein, e.g. with the Fc domain of a human antibody. The small synthetic molecular antagonist of the HMGB1 interaction with RAGE preferably has a molecular weight of less than 1000 Dalton. The small synthetic molecular antagonist preferably inhibits the interaction of RAGE with the non-acetylated form or/and with the acetylated form of HMGB1 and with the non-acetylated form or/and with the acetylated form of HMGB1 homologous proteins, particularly HMGB2, HMGB3, HMG-1L10, HMG-4L or/and SP100-HMG.

Furthermore, the further agent may be an HMGB1 antagonist/inhibitor, e.g. an antibody against HMGB1, particularly against the HMGB1 Box-B or a fragment of HMGB1 which has antagonistic activity, e.g. a Box-A fragment. Suitable HMGB1 antagonists and inhibitors are disclosed in U.S. Pat. No. 6,468,533, WO 02/074337 and US 2003/144201, which are incorporated herein by reference. The HMGB1 antagonist/inhibitor is preferably an antagonist/inhibitor of the non-acetylated or/and acetylated form of HMGB1.

The further agent used in combination with K-252a or/and at least one kinase inhibitor or/and derivatives thereof may also be an inhibitor of the interaction of a Toll-like receptor (TLR), e.g. of TLR2, TLR4, TLR7, TLR8 or/and TLR9, with HMGB1, which inhibitor is preferably a monoclonal or polyclonal antibody, a nucleic acid or nucleic acid analogue capable of inhibiting TLR expression, e.g. an antisense molecule, a ribozyme or a RNA interference molecule, or a synthetic molecule preferably having a size of less than 1000 Dalton. The inhibitor may be a known inhibitor of a Toll-like receptor, in particular of TLR2, TLR4, TLR7, TLR8 or/and TLR9. The inhibitor preferably inhibits the interaction of the Toll-like receptor with the non-acetylated form or/and the acetylated form of HMGB1 and with the non-acetylated form or/and with the acetylated form of HMGB1 homologous proteins, in particular HMGB2, HMGB3, HMG-1L10, HMG-4L or/and SP100-HMG.

In still another embodiment, the further agent used in combination with K-252a or/and at least one kinase inhibitor or/and derivatives thereof is the functional N-terminal lectin-like domain (D1) of thrombomodulin. The D1 domain of thrombomodulin is able to intercept the non-acetylated form and/or the acetylated form of released HMGB1 and of released HMGB1 homologous proteins, in particular HMGB2, HMGB3, HMG-1L10, HMG-4L or/and SP100-HMG, preventing thus their interaction with RAGE and Toll-like receptors. The D1 domain of thrombomodulin may be native or mutated in order to make it resistant to proteases.

The further agent may also be a synthetic double-stranded nucleic acid or nucleic acid analogue molecule with a bent shape structure, particularly a double-stranded bent DNA, PNA or DNA/PNA chimera or hybrid or a double-stranded cruciform DNA, PNA or DNA/PNA chimera or hybrid structure, capable of binding to the HMGB1 protein. Preferred nucleic acids and nucleic analogue molecules are disclosed in a co-owned and co-pending international patent application no. PCT/EP2005/007198 filed on 4 Jul. 2005 (claiming the priority of U.S. provisional application No. 60/584,678 filed on 2 Jul. 2004), which are incorporated herein by reference. The synthetic double-stranded nucleic acid or nucleic acid analogue molecule with a bent shape structure is preferably capable of binding to the non-acetylated or/and to the acetylated form of HMGB1 and the non-acetylated or/and the acetylated form of HMGB1 homologous proteins, in particular HMGB2, HMGB3, HMG-1L10, HMG4L or/and SP100-HMG.

The K-252a or/and at least one kinase inhibitor or/and a derivative thereof is/are usually administered as a pharmaceutical composition, which additionally comprises pharmaceutically acceptable carriers, diluents and/or adjuvants.

The administration may be carried out by known methods, e.g. by injection, in particular by intravenous, intramuscular, transmucosal, subcutaneous or intraperitoneal injection and/or by oral, topical, nasal, inhalation, aerosol and/or rectal application, etc. The administration may be local or systemic.

Therefore, a further aspect of the present invention is a pharmaceutical composition comprising an effective amount of
(i) K-252a or/and
(ii) at least one kinase inhibitor, or/and a salt or a derivative of (i) or/and (ii) as an active ingredient for the treatment of HMGB1-associated pathologies and pharmaceutically acceptable carriers, diluents and/or adjuvants. The pharmaceutical composition of the present invention is preferably suitable for the treatment of pathologies associated with the non-acetylated or/and the acetylated form of HMGB1 and/or of HMGB1 homologous proteins.

It is preferred that the pharmaceutical composition of the present invention comprises at least one kinase inhibitor, alone or in combination with K-252a, which kinase inhibitor is selected from tyrosine kinase inhibitors or/and phosphokinase inhibitors. A preferred tyrosine kinase inhibitor in the pharmaceutical composition of the present invention is an inhibitor of TrkA, TrkB, TrkC or/and an inhibitor of the subfamily of tyrosine kinase receptors including Ron, c-Met and Sea receptors. A preferred phosphokinase inhibitor in the pharmaceutical composition of the present invention is an inhibitor of PKA, PKC, or/and PKG. Another preferred kinase inhibitor in the; pharmaceutical composition of the present invention is an inhibitor of Raf kinase, Ras kinase, CaM kinase, MLC kinase, MAP kinase, MEK, ERK, JUN kinase or/and PI3Kγ. The at least one kinase inhibitor in the pharmaceutical composition of the present invention is preferably a known kinase inhibitor, such as a known kinase inhibitor as described above.

In a further preferred embodiment, the pharmaceutical composition of the present invention comprising K-252a or/and at least one kinase inhibitor comprises a further agent as defined above.

The exact formulation, route of administration and dosage can be chosen by the individual physician in view of the patient's conditions. Administration may be achieved in a single dose or repeated doses at intervals. Dosage amount and interval may be adjusted individually in order to provide the therapeutical effect which results in amelioration of symptoms 0 or a prolongation of the survival in a patient. The actual amount of composition administered will, of course, be dependent on the subject being treated, on the subject's weight, the severity of the affliction, the manner of administration and the judgement of the prescribing physician. A suitable daily dosage will be between 0.001 to 10 mg/kg, particularly 0.1 to 5 mg/kg.

The pharmaceutical composition of the present invention may be used for diagnostic or for therapeutic applications. For diagnostic applications, the compound may be present in a labelled form, e.g. in a form containing an isotope, e.g. a radioactive isotope or an isotope which may be detected by o nuclear magnetic resonance. A preferred therapeutic application is blocking, retarding or reducing connective tissue regeneration. K-252a (i) or/and at least one kinase inhibitor (ii), or/and a salt or a derivative of (i) or/and (ii) may be administered as a free compound and/or reversibly immobilized on the surface of the medical device. For this purpose, a medical device may be reversibly loaded with the active ingredient and, optionally, further agents, in particular by binding, embedding and/or absorbing the medicament molecules onto the surface of the medical device or on a coating layer on the surface of the medical device. After contacting the medical device with body fluid or body tissue, the reversibly immobilised compounds are liberated. Consequently, the coated medical devices act as drug delivery devices eluting the medicament, whereby the drug delivery kinetics can be controlled, providing an immediate release or a controlled, delayed or sustained drug delivery, for example. For a controlled, delayed or sustained release, the active agent can be embedded into nano- or microcapsules or a matrix coating, in particular a polymer matrix coating can be applied on a medical device, such as a stent. Coating technologies of medical devices are well known to the person skilled in the art.

Therefore, a further aspect of the present invention relates to a medical device reversibly coated or embedded with (i) K-252a or/and (ii) at least one kinase inhibitor, or/and a salt or a derivative of (i) or/and (ii). Preferably, the medical device is selected from surgical instruments, implants, catheters or stents, e.g. stents for angioplasty. Most preferably, the medical device according to the invention is a drug-eluting stent (DES).

Further, the present invention is explained in more detail in the following Figures and Examples.

### BRIEF DESCRIPTION OF THE DRAWINGS

FIG. 1—Activity of K-252a in a chemotaxis assay.

Inhibitory activity of K-252a on bovine aorta smooth muscle cells (BASMC) in a typical migration (chemotaxis) assay performed using modified Boyden chambers and two different chemoattractants: HMGB1 and fMLP (formyl methionine leucine phenylalanine peptide—or fMetLeuPhe—a specific chemoattractant of leucocytes). K-252a actively and concentration-dependently (in a nanomolar range) antagonizes HMGB1-induced BASMC cell migration, while it does not interfere with cell migration induced by fMLP, whatever the concentration tested.

FIG. 2—Activity of K-252a in a proliferation assay.

K-252a inhibitory activity on HMGB1-induced bovine aorta smooth muscle cell (BASMC) proliferation. K-252a antagonizes BASMC cell proliferation at all the concentrations tested in a time-dependent fashion and in a nM range.

FIG. 3—circular dichroism of HMGB1 in the presence of K-252a.

FIG. 4—Inhibition by K-252a of mortality by LPS-induced endotoxemia. Treatment with K-252a shows a clear reversal of lethality induced by LPS in mice.

### EXAMPLES

### 1. Chemotaxis Assay

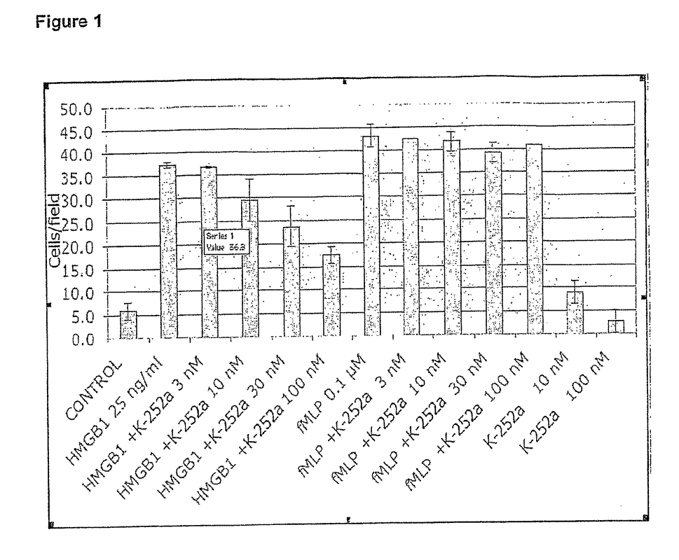

Chemotaxis assays were performed using a well known and validated protocol (1). Modified Boyden chambers were used with filters having 5-8 μm pore size and treated with collagen I (100 μg/ml in 0.5 M acetic acid) and fibronectin (10 μg/ml, Roche). BASMC (bovine aorta smooth muscle cells) were cultured in serum-free DMEM and a sample of 20,000-40,000 cells was added to the upper well of a Boyden chamber. K-252a was dissolved and diluted in the same serum-free medium and added to the lower well of the chamber. HMGB-1 (from calf thymus) concentration was 25 ng/ml, that one of fMLP was 0.1 μM while K-252a was 3, 10, 30, 100 nM. Overnight cell migration was allowed at 37+/−0.5° C., then cells were scraped off and filters were fixed in methanol and stained in a solution of 10% crystal violet in 20% methanol. All experiments were performed at least twice in triplicate. Results are the mean+/−SD of the number of cells counted in 10 high power fields per filters and expressed as folds over control. Random cell migration, i.e. migration in the absence of chemoattractant, was given the arbitrary value of 100%. Statistical analysis was performed using Student's t test for pairwise comparisons of treatment, or an ANOVA model for the evaluation of treatments with increasing concentrations of a reagent. The results are shown in FIG. 1.

A chemotaxis assay as described above can be similarly performed using BAEC cells. Corresponding results are obtained.

### 2. Proliferation Assay

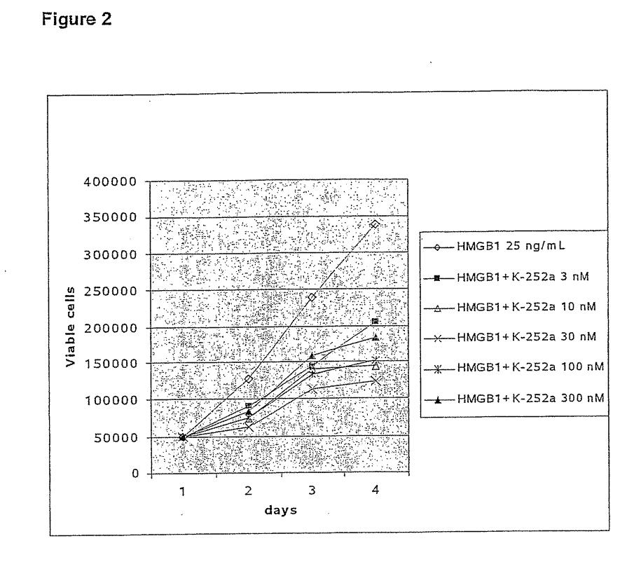

Proliferation assays were performed using an already described and validated method (1). BASMC cells (bovine aorta smooth muscle cells) were seeded in 6-well plates (105 cells/well) and grown in RPMI medium supplemented with 20% FCS. After 24 h, the medium was replaced with serum-free RPMI and cell were then starved for 16 hours to synchronize the cell population. Vehicle (negative control or basal proliferation) or 25 ng/ml (1 nM) of HMGB1 (bacterially made) were added in the presence or in the absence of 3, 10, 30, 100 or 300 nM K-252a (dissolved and diluted in serum-free medium). Each experimental point represents the mean+/−SD of triplicate determinations. The experiment was repeated three times. BASMC cell proliferation was determined by detaching the cells from the plate at the indicated times (on days 1, 2, 3, and 4 of culturing) and counting the Trypan-blue excluding cells under the microscope. The results are shown in FIG. 2.

A proliferation assay as described above can be similarly performed using BAEC cells. Corresponding results are obtained.

### 3. Protein Binding Experiments by Circular Dichroism (CD)

To check for protein binding of K-252a, a CD study was performed. All CD spectra were collected on a Jasco J710 spectropolarimeter equipped with a NesLab RTE111 thermal controller unity, using a quartz cylindrical cuvette with a 1 cm path length (Jasco). A scan speed of 20 nm/min, a bandwidth of o 1 nm, and a resolution of 1 nm was always used.

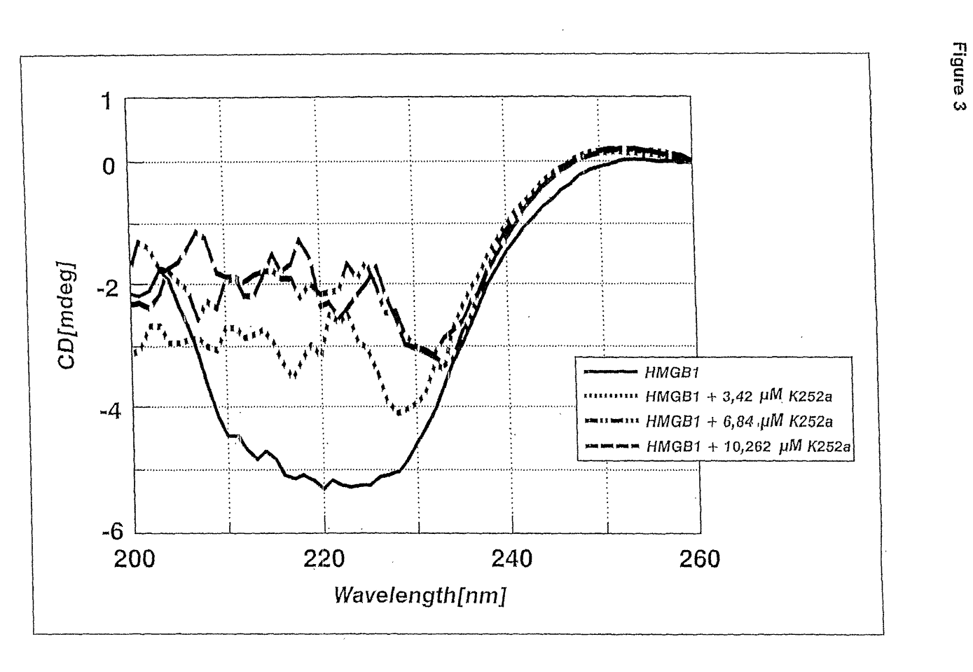

The addition of K-252a in concentrations of 3.42 μM, 6.84 μM and 10.26 μM to HMGB1 induces a great impact on the CD of HMGB1 in the range of 200 to about 235 nm (see FIG. 3), but not in the range of about 235 to 260 nm. s The effect in the range of 200 to about 235 nm could, however, be attributed to the denaturating action of the solvent of K-252a (dimethylformamide, DMF) or to the interaction between K-252a and bacterial contaminants of HMGB1. The spectra are heavily disturbed more than likely due to DMF.

Assuming that a 1:1 stoichiometry binding takes place between HMGB1 and K-252a, the apparent Kd of the complex should be approximately 2 μM, a value which characterizes an extremely weak interaction. Therefore, direct interaction can not be considered the mechanism by which K-252a inhibits HMGB1-induced cell proliferation and migration. Fluorescence assays seem 5 to confirm the absence of a direct interaction between K-252a and HMGB1.

K-252a is an inhibitor of tyrosine kinases (TrkA, TrkB, TrkC), of phosphokinases (PKA, PKC, PKG), and of further kinases (Raf kinase, Ras kinase, CaM kinase, MLC kinase, MAP kinase, MEK, ERK, JUN kinase, o PI3Kγ). More than likely, K-252a does not inhibit HMGB1 at the receptor (RAGE) level either, but it may interfere with TrkA and/or with one of the kinases downstream of the HMGB1 interaction with RAGE or with Toll receptors.

### 4. Reversal by K-252a of LPS-Induced Endotoxemia in Mice

Thirty-two male 6 to 7-week-old BALB/c mice were purchased from Charles River (Calco, Italy) and allowed to acclimatise for one week before use. On the day of the experiment, all mice were given an LD70-90 (10.5 mg/kg i.p. in the right inguinal region) of lipopolysaccharide (LPS from Escherichia coli, strain 0111:B4 SIGMA, Lot 034154105), dissolved in 0.9% sterile saline. 15 min before LPS injection and 2.12 and 24 h after LPS administration, 16 mice received K-252a (6.7 mg/kg i.p., 10 ml/kg, in the left inguinal region) dissolved in DMSO: sterile saline (8:92 v/v). The remaining 16 mice received the same volume of the vehicle alone (controls). Mice were observed for 7 consecutive days at least twice a day and deaths were recorded.

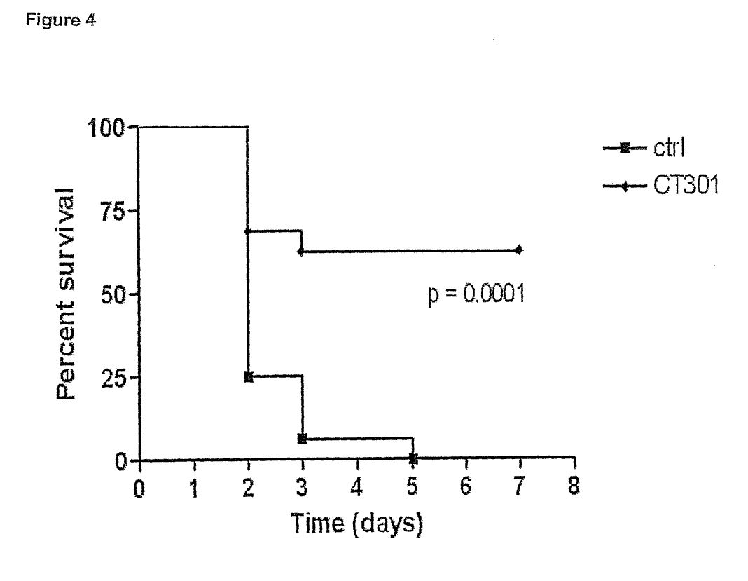

The results are shown in FIG. 4. At the end of the observation period (7 days), 10 out of 16 mice (62.5%) treated with K-252a were still alive, while the last control mouse was found dead already in the course of the fifth day. Statistical analysis (Kaplan-Meier Survival Analysis) gives a “P” value of 0.0001.

### 5. Kinase Inhibition Profiling Study

An analysis has been conducted with the aim of measuring the inhibitory activities of K-252a against 67 protein kinases at a concentration of 200 nM. In particular, the target kinases were Tyrosine kinases and Serine/Threonine kinases.

5.1 • Methods and Materials

1. Test Compounds

Test compound K-252a was stored in at −20 C in the dark until use. Reference compounds for assay control listed below were used.

| Reference compounds | Concentration (nM) | Kinase |
|---|---|---|
| Staurosporine | 0.3-10000 | Tyrosine kinases |
| | | Other Serine/Threonine |
| | | kinases |
| 5-Iodotubericidin | 10000 | ERK1, ERK2 |
| JNK Inhibitor II | 300 | JNK1, JNK2 |
| SB202190 | 300 | p38α, p38β |

2. Preparation of Test Compound Solution

Each test compound was weighed and dissolved in DMSO to make 1 mM stock solution. The stock solution was diluted with DMSO to achieve 20 μM. The compound solution was diluted with the assay buffer to achieve 0.8 μM.

Preparation of reference compound was conducted similar method to the test compounds.

3. Kinases Used

Tyrosine Kinases

| Kinase | Description |
|---|---|
| ABL | Human ABL [2-1130 amino acids of accession number |
| | CAA34438] was expressed as N-terminal His-tagged |
| | protein (126 kDa) using baculovirus expression system. |
| | His-tagged ABL was purified by using Ni-NTA affinity |
| | chromatography. |
| ARG | Human ARG [2-1182 amino acids of accession number |
| | NP_009298] was expressed as N-terminal |
| | His-tagged protein (129 kDa) using baculovirus |
| | expression system. His-tagged ARG was purified by using |
| | Ni-NTA affinity chromatography. |
| TNK1 | Kinase domain (KD) of human TNK [110-394 amino |
| | acids of accession number AAC99412] was |
| | expressed as N-terminal GST-fusion protein |
| | (58 kDa) using baculovirus expression system. |
| | GST-TNK1 KD was purified by using glutathione |
| | sepharose chromatography and gel filtration |
| | chromatography. |
| ALK | Cytoplasmic domain of human ALK [1089-1620 |
| | amino acids of accession number AAC51104] |
| | was expressed as N-terminal His-tagged protein |
| | (62 kDa) using baculovirus expression system. |
| | His-tagged ALK was purified by using Ni-NTA |
| | affinity chromatography. Purified His-tagged |
| | ALK was digested by recombinant His-TEV protease, |
| | and His-tag free ALK (ca. 61 kDa) was collected |
| | as flow-through fraction from Ni-NTA affinity |
| | chromatography. |
| AXL | Human AXL [1-885 amino acids of accession |
| | number NP_001690] was expressed |
| | as N-terminal His-tagged protein (51 kDa) using |
| | baculovirus expression system. His-tagged AXL |
| | was purified by using Ni-NTA affinity |
| | chromatography and anion exchange chromatography. |
| MER | Human MER [528-999 amino acids of accession |
| | number NP_006334] was expressed |
| | as N-terminal His-tagged protein (56 kDa) using |
| | baculovirus expression system. His-tagged MER |
| | was purified by using Ni-NTA affinity chromatography. |
| CSK | Human CSK [1-450 amino acids of accession |
| | number NP_004374] was expressed as |
| | N-terminal His-tagged protein (54 kDa) using baculovirus |
| | expression system. His-tagged CSK was purified by |
| | using Ni-NTA affinity chromatography. Purified His- |
| | tagged CSK was digested by recombinant His-TEV |
| | protease, and His-tag free CSK (ca. 50 kDa) was |
| | collected as flow-through fraction from Ni-NTA |
| | affinity chromatography. |
| EGFR | Cytoplasmic domain of human EGFR [669-1210 amino |
| | acids of accession number NP_005219] |
| | was expressed as N-terminal His-tagged protein |
| | (64 kDa) using baculovirus expression system. His- |
| | tagged EGFR was purified by using Ni-NTA affinity |
| | chromatography. |
| HER2 | Cytoplasmic domain of human HER2 [676-1255 amino |
| | acids of accession number NP_004439] |
| | was expressed as N-terminal His-tagged protein (67 |
| | kDa) using baculovirus expression system. His-tagged |
| | HER2 was purified by using Ni-NTA affinity |
| | chromatography. |
| HER4 | Cytoplasmic domain of human HER4 [676-1308 amino |
| | acids of accession number NP_005226] |
| | was expressed as N-terminal His-tagged protein (75 |
| | kDa) using baculovirus expression system. His-tagged |
| | HER4 was purified by using Ni-NTA affinity |
| | chromatography. |
| EphA2 | Cytoplasmic domain of human EphA2 [572-976 amino |
| | acids of accession number NP_004422] |
| | was expressed as N-terminal His-tagged protein (49 |
| | kDa) using baculovirus expression system. His-tagged |
| | EphA2 was purified by using Ni-NTA affinity |
| | chromatography. |
| EphB2 | Cytoplasmic domain of human EphB2 [581-987 amino |
| | acids of accession number NP_004433] |
| | was expressed as N-terminal His-tagged protein (49 |
| | kDa) using baculovirus expression system. His-tagged |
| | EphB2 was purified by using Ni-NTA affinity |
| | chromatography. |
| EphB4 | Cytoplasmic domain of human EphB4 [577-987 amino |
| | acids of accession number NP_004435] |
| | was expressed as N-terminal His-tagged protein (49 |
| | kDa) using baculovirus expression system. His-tagged |
| | EphB4 was purified by using Ni-NTA affinity |
| | chromatography. |
| FAK | Human FAK [1-1052 amino acids of accession number |
| | NP_722560] was expressed as N-terminal |
| | His-tagged protein (122 kDa) using baculovirus |
| | expression system. His-tagged FAK was purified by |
| | using Ni-NTA affinity chromatography. |
| FGFR1 | Cytoplasmic domain of human FGFR1 [396-820 amino |
| | acids of accession number NP_056934] |
| | was expressed as N-terminal His-tagged protein (51 |
| | kDa) using baculovirus expression system. His-tagged |
| | FGFR1 was purified by using Ni-NTA affinity |
| | chromatography. |
| FGFR2 | Cytoplasmic domain of human FGFR2 [399-821 amino |
| | acids of accession number NP_000132] |
| | was expressed as N-temrinal His-tagged protein (51 |
| | kDa) using baculovirus expression system. His-tagged |
| | FGFR2 was purified by using Ni-NTA affinity |
| | chromatography. |
| IFG1R | Cytoplasmic domain of human IGF1R [959-1367 amino |
| | acids of accession number NP_000866] |
| | was expressed as N-terminal His-tagged protein (50 |
| | kDa) using baculovirus expression system. His-tagged |
| | IGF1R was purified by using Ni-NTA affinity |
| | chromatography. |
| INSR | Kinase domain (KD) of human INSR [1005-1310 amino |
| | acids of accession number NP_000199] |
| | was expressed as N-terminal His-tagged protein (38 |
| | kDa) using baculovirus expression system. His-tagged |
| | INSR KD was purified by using Ni-NTA affinity |
| | chromatography. Purified His-tagged INSR KD was |
| | digested by recombinant His-TEV protease, and His- |
| | tag free INSR KD (ca. 37 kDa) was collected as |
| | flow-through fraction from Ni-NTA affinity |
| | chromatography. |
| JAK1 | Kinase domain (KD) of human JAK1 [838-1142 amino |
| | acids of accession number NP_002218] |
| | was expressed as N-terminal GST-fusion protein (62 |
| | kDa) using baculovirus expression system. GST-JAK1 KD |
| | was purified by using glutathione sepharose |
| | chromatography. |
| JAK2 | Kinase domain (KD) of human JAK2 [826-1132 amino |
| | acids of accession number NP_004963] |
| | was expressed as N-terminal His-tagged protein (39 |
| | kDa) using baculovirus expression system. His-tagged |
| | JAK2 KD was purified by using Ni-NTA affinity |
| | chromatography and anion exchange chromatography. |
| JAK3 | Kinase domain (KD) of human JAK3 [795-1124 amino |
| | acids of accession number NP_000206] |
| | was expressed as N-terminal His-tagged protein (41 |
| | kDa) using baculovirus expression system. His-tagged |
| | JAK3 KD was purified by using Ni-NTA affinity |
| | chromatography and gel filtration chromatography. |
| TYK2 | Kinase domain (KD) of human TYK2 [871-1187 amino |
| | acids of accession number NP_003322 was |
| | expressed as N-terminal GST-fusion protein (63 kDa) |
| | using baculovirus expression system. GST-TYK2 KD |
| | was purified by using glutathione sepharose |
| | chromatography. |
| MET | Human MET [956-1390 amino acids of accession number |
| | CAB56793] was expressed as N-terminal His-tagged |
| | protein (52 kDa) using baculovirus expression system. |
| | His-tagged MET was purified by using Ni-NTA affinity |
| | chromatography and anion exchange chromatography. |
| RON | Cytoplasmic domain of human RON [979-1400 amino |
| | acids of accession number NP_002438] |
| | was expressed as N-terminal His-tagged protein |
| | (51 kDa) using baculovirus expression system. His- |
| | tagged RON was purified bby using Ni-NTA affinity |
| | chromatography. |
| FLT3 | Human FLT3 [564-993 amino acids of accession number |
| | NP_004110)] was expressed as N-terminal |
| | His-tagged protein (53 kDa) using baculovirus expression |
| | system. His-tagged FLT3 was purified by using Ni-NTA |
| | affinity chromatography. |
| FMS(CSFR) | Cytoplasmic domain of human FMS [538-972 amino |
| | acids of accession number NP_005202] was |
| | expressed as N-terminal His-tagged protein (52 kDa) |
| | using baculovirus expression system. His-tagged FMS |
| | was purified by using Ni-NTA affinity chromatography. |
| KIT | Human KIT [544-976 amino acids of accession number |
| | NP_000213] was expressed as N-terminal His- |
| | tagged protein (52 kDa) using baculovirus expression |
| | system. His-tagged KIT was purified by using Ni-NTA |
| | affinity chromatography. |
| PDGFRα | Cytoplasmic domain of human PDGFRα [550-1089 |
| | amino acids of accession number NP_006197] |
| | was expressed as N-terminal His-tagged protein (65 kDa) |
| | using baculovirus expression system. His-tagged PDGFRα |
| | was purified by using Ni-NTA affinity chromatography. |
| PDGFRβ | Cytoplasmic domain of human PDGFRβ [557-1106 amino |
| | acids of accession number NP_002600] was |
| | expressed as N-terminal His-tagged protein (65 kDa) |
| | using baculovirus expression system. His-tagged |
| | PDGFRβ was purified by using Ni-NTA affinity |
| | chromatography. |
| RET | Cytoplasmic domain of human RET [658-1114 amino acids |
| | of accession number NP_000314] was |
| | expressed as N-terminal His-tagged protein 56 kDa) |
| | using baculovirus expression system. His-tagged RET was |
| | purified by using Ni-NTA affinity chromatography. |
| BLK | Human BLK [1-505 amino acids of accession number |
| | NP_001706] was expressed as N-terminal |
| | His-tagged protein (61 kDa) using baculovirus |
| | expression system. His-tagged BLK was purified by using |
| | Ni-NTA affinity chromatography. |
| BRK | Human BRK [2-450 amino acids of accession number |
| | NP_005966] was expressed as N-terminal |
| | His-tagged protein (49 kDa) using baculovirus |
| | expression system. His-tagged BRK was purified by using |
| | Ni-NTA affinity chromatography. |
| FGR | Human FGR [1-529 amino acids of accession number |
| | AAA52451] was expressed as N-terminal His-tagged |
| | protein (63 kDa) using baculovirus expression system. |
| | Purified His-tagged FGR was digested by recombinant |
| | His-TEV protease, and His-tag free FGR (ca. 60 kDa) was |
| | collected as flow-through fraction from Ni-NTA affinity |
| | chromatography. |
| FYN | Human FYN [1-537 amino acids of accession number |
| | NP_002028] was expressed as N-terminal |
| | His-tagged protein (64 kDa) using baculovirus expression |
| | system. His-tagged FYN was purified by using Ni-NTA |
| | affinity chromatography. |
| HCK | Human HCK [73-526 amino acids of accession number |
| | NP_002101] was expressed as N-terminal |
| | His-tagged protein (60 kDa) using baculovirus |
| | expression system. His-tagged HCK was purified by using |
| | Ni-NTA affinity chromatography. |
| LCK | Human LCK [1-509 amino acids of accession number |
| | NP_005347] was expressed as N-terminal |
| | His-tagged protein (61 kDa) using baculovirus |
| | expression system. His-tagged LCK was purified by using |
| | Ni-NTA affinity chromatography. |
| LYNa | Human LYNa [1-512 amino acids of accession number |
| | NP_002341] was expressed as N-terminal His- |
| | tagged protein (62 kDa) using baculovirus expression |
| | system. His-tagged LYNa using Ni-NTA affinity |
| | chromatography. |
| LYNb | Human LYNb [1-491 amino acids of accession number |
| | NP_02341] was expressed as N-terminal His- |
| | tagged protein (59 kDa) using baculovirus expression |
| | system. His-tagged LYNb was purified by using Ni-NTA |
| | affinity chromatography. |
| SRC | Human SRC [1-536 amino acids of accession number |
| | NP_005408] was expressed as N-terminal |
| | His-tagged protein (63 kDa) using baculovirus expression |
| | system. His-tagged SRC was purified by using Ni-NTA |
| | affinity chromatography. |
| SRM | Kinase domain (KD) of human SRM [215-489 amino acids |
| | of accession number NP_543013] was |
| | expressed as N-terminal His-tagged protein (34 kDa) |
| | using baculovirus expression system. His-tagged SRM KD |
| | was purified by using Ni-NTA affinity chromatography. |
| YES | Human YES [1-543 amino acids of accession number |
| | NP_005424] was expressed as N-teminal |
| | His-tagged protein (64 kDa) using baculovirus |
| | expression system. His-tagged YES was purified by |
| | using Ni-NTA affinity chromatography. |
| TRKA | Cytoplasmic domain of human TRKA [436-790 amino |
| | acids of accession number AAA36770] was expressed |
| | as N-terminal His-tagged protein (43 kDa) using |
| | baculovirus expression system. His-tagged TRKA was |
| | purified by using Ni-NTA affinity chromatography |
| | and gel filtration chromatography. |
| TRKB | Cytoplasmic domain of human TRKB [456-822 amino |
| | acids of accession number AAC51371] was expressed |
| | as N-terminal His-tagged protein (45 kDa) using |
| | baculovirus expression system. His-tagged TRKB was |
| | purified by using Ni-NTA affinity chromatography. |
| TRKC | Cytoplasmic domain of human TRKC [456-839 amino |
| | acids of accession number AAB33111] was expressed |
| | as N-terminal His-tagged protein (47 kDa) using |
| | baculovirus expression system. His-tagged TRKC was |
| | purified by using Ni-NTA affinity chromatograph |
| | and gel filtration chromatography. |
| FLT1 | Cytoplasmic domain of human FLT1 [781-1338 amino |
| | acids of accession number NP_002010] was |
| | expressed as N-terminal His-tagged protein (67 kDa) |
| | using baculovirus expression system. His-tagged FLT1 |
| | was purified by using Ni-NTA affinity chromatography. |
| KDR | Cytoplasmic domain of human KDR [790-1356 amino |
| | acids of accession number NP_002244] was |
| | expressed as N-terminal His-tagged protein (66 kDa) |
| | using baculovirus expression system. His-tagged KDR was |
| | purified by using Ni-TA affinity chromatography. |

Serine/Threonine Kinases

| Kinase | Description |
|---|---|
| PKACα | Human PKACα [1-351 amino acids of accession number |
| | NP_002721] was expressed as N-terminal His-tagged |
| | protein (44 kDa) using baculovirus expression system. |
| | His-tagged PKACα was purified by using Ni-NTA |
| | affinity chromatography. |
| PKCα | Human PKCα [1-672 amino acids of accession number |
| | NP_002728] was expressed as N-terminal His-tagged |
| | protein (80 kDa) using baculovirus expression system. |
| | His-tagged PKCα was purified by using Ni-NTA |
| | affinity chromatography. |
| PKCε | Human PKCε [1-737 amino acids of accession number |
| | NP_005391] was expressed as N-terminal His-tagged |
| | protein (88 kDa) using baculovirus expression system. |
| | His-tagged PKCε was purified by using Ni-NTA |
| | affinity chromatography. |
| PKCγ | Human PKCγ [1-697 amino acids of accession number |
| | NP_002730] was expressed as N-terminal His-tagged |
| | protein (83 kDa) using baculovirus expression system. |
| | His-tagged PKCγ was purified by using Ni-NTA |
| | affinity chromatography. |
| CaMK4 | Human CaMK4 [1-473 amino acids of accession number |
| | NP_001735] was expressed as N-terminal His-tagged |
| | protein (54 kDa) using baculovirus expression system. |
| | His-tagged CaMK4 was purified by using Ni-NTA |
| | affinity chromatography. |
| CaMK2α | Human CaMK2α [1-478 amino acids of accession |
| | number NP_741960] was expressed as N-terminal |
| | His-tagged protein (57 kDa) using baculovirus expression |
| | system. His-tagged CaMK2α was purified by using Ni- |
| | NTA affinity chromatography. |
| CHK1 | Human CHK1 [1-476 amino acids of accession number |
| | NP_001265] was expressed as N-terminal His-tagged |
| | protein (57 kDa) using baculovirus expression system. |
| | His-tagged CHK1 was purified by using Ni-NTA |
| | affinity chromatography. |
| MAPKAPK2 | Human MAPKAPK2 [1-400 amino acids of accession |
| | number NP_116584] was expressed as N-terminal |
| | His-fusion protein (49 kDa) using baculovirus expression |
| | system. His-tagged MAPKAPK2 was purified by using |
| | Ni-NTA affinity chromatography and activated with |
| | p38β. Activated MAPKAPK2 was purified using gel |
| | filtration chromatography. |
| CHK2 | Human CHK2 [1-543 amino acids of accession number |
| | NP_009125] was expressed as N-terminal His-tagged |
| | protein (65 kDa) using baculovirus expression system. |
| | His-tagged CHK2 was purified by using Ni-NTA |
| | affinity chromatography. |
| CDK2/cyclinA | Human CDK2 [1-298 amino acids of accession |
| | number NP_001789] and CyclinA2 [1-432 amino |
| | acids of accession number NP_001228] was co- |
| | expressed as N-terminal His-tagged protein using |
| | baculovirus expression system. His-tagged CDK2 was |
| | purified by using Ni-NTA affinity chromatography. The |
| | apparent MW if His tagged CDK2 was approximately 37 |
| | kDa. |
| Erk1 | Human Erk1 containing 379 amino acids was expressed |
| | as N-terminal His-tagged (43 kDa) in E. coli expression |
| | and activated with MAP2K1. |
| | Erk1 was purified using Ni-NTA affinity |
| | chromatography, Gel permeation chromatography and |
| | Q sepharose. |
| Erk2 | Human Erk2 containing 360 amino acids was expressed |
| | as N-terminal His-tagged (41 kDa) in E. coli expression |
| | and activated with MAP2K1. |
| | Erk2 was purified using Ni-NTA affinity |
| | chromatography, Gel permeation chromatography and |
| | Q sepharose. |
| JNK1 | Human JNK1 containing 364 amino acids was expressed |
| | as C-terminal His-tagged (40 kDa) in E. coli expression |
| | and activated with MAP2K7. |
| | JNK1 was purified using Ni-NTA affinity |
| | chromatography, SP sepharose and gel permeation |
| | chromatography. |
| JNK2 | Human JNK2 containing 424 amino acids was expressed |
| | as C-terminal His-tagged (47 kDa) in E. coli expression |
| | and activated with MAP2K7. |
| | JNK2 was purified using Ni-NTA affinity |
| | chromatography, SP sepharose and Gel permeation |
| | chromatography, |
| p38α | Human p38α [9-352 amino acids of accession |
| | number NP_620581] was expressed as N- |
| | terminal GST-fusion protein (66 kDa) using the E. coli |
| | expression system. GST-tagged p38α was purified by |
| | using glutathione sepharose chromatography and |
| | activated with MAP2K6. Activated p38α was purified |
| | using glutathione sepharose chromatography. |
| p38β | Human p38β containing 334 amino acids (38 kDa) |
| | was expressed as N-terminal GST-tagged in E. coli and |
| | activated with MAP2K6. P38β was purified using |
| | glutathione affinity chromatography |
| AurA | Human AurA [1-403 amino acids of accession number |
| | NP_940835] was expressed as N-terminal |
| | GST-fusion protein (73 kDa) using baculovirus |
| | expression system. GST-AurA was purified by using |
| | glutathione sepharose chromatography. |
| IKKβ | Human IKKβ with 662 amino acids were expressed |
| | as N-terminal His-tagged (76 kDa) in insect cells. |
| | IKKβ was purified using Ni-NTA affinity |
| | chromatography, gel permeation chromatography, and |
| | Heparin chromatography. |
| MAP2K3 | Constitutive active human MAP2K3(2E) [S218E, |
| | T222E] derived from wild type [347 amino acids |
| | of accession number NP_659731] was |
| | expressed as N-terminal His-fusion protein (42 kDa) |
| | using baculovirus expression system. His-tagged |
| | MAP2K3 (2E) was purified by using Ni-NTA affinity |
| | chromatography. |
| NAP2K7 | Constitutive active human MAP2K7(3E) [S271E, |
| | T275E, S277E] derived from wild type [419 amino acids |
| | of accession number NP_660186] was |
| | expressed as N-terminal GST-fusion protein (70 kDa) |
| | using the E. coli expression system. |
| | GST-MAP2K7(3E) was purified by using glutathione |
| | sepharose chromatography. |
| IRAK4 | Kinase domain (KD) of Human IRAK4 [172-460 amino |
| | acids of accession number NP_057207] |
| | was expressed as N-terminal GST-fusion protein 59 kDa) |
| | using baculovirus expression system. GST-IRAK4 KD |
| | was purified by using glutathione sepharose |
| | chromatography. |

4. Assay Reagents

ELISA

| Component | Description |
|---|---|
| Assay buffer | 15 mM Tris-HCl, pH 7.5, 0.01% Tween-20, 2 mM |
| | DTT |
| Assay plate | Streptavidine-coated yellow plate, 96-well |
| | (PerkinElmer Inc. #AAAND-0005) |
| Blocking buffer | 50 mM Tris-HCl, pH 7.5, 150 mM NaCl, 0.02% |
| | Tween-20, 0.1% BSA |
| Detection antibody | Tyrosine kinase |
| | HRP conjugated p-Tyr (PY20) |
| | (Santa Cruz Biotechnology Inc. #sc-508 HRP) |
| | JNK1, JNK2 |
| | Monoclonal Anti-Phospho-ATF2 (Phosphothreo- |
| | nine, 69, 71) |
| | Clone ATF-22P (SIGMA, A-4095) |
| | goat Anti-Mouse IgG Ab HRP conjugate (Zymed |
| | #62-6520) |
| HRP substrate | TMB solution |
| | (MOSS Inc. #TMBE-1000) |
| Plate reader | SpectraMAX |
| | (Molecular Devices Corp.) |

IMAP Assay

| Component | Description |
|---|---|
| Assay buffer | 20 mM HEPES, pH 7.4, 0.01% Tween-20, 2 mM DTT |
| Assay plate | 384 well black plate (Corning, 3710) |
| Detection reagent | IMAP Screening Express kit |
| | (Molecular Devices Corp., R8073) |
| Plate reader | Analyst AD (Molecular Devices Corp.) |

AlphaScreen Assay

| Componeent | Description |
|---|---|
| Assay buffer | MAP2K3 |
| | 25 mM HEPES, pH 7.4, 0.01% Tween-20, 2 mM |
| | DTT |
| | MAP2K7 |
| | 50 mM HEPES, pH 7.4, 0.01% Tween-20, 1 mM |
| | DTT |
| Assay plate | OptiPlate-384, White (PerkinElmer #6007290) |
| Detection antibody | MAP2K3 |
| | Biotinylated anti-phospho p38 antibody |
| | (Cell Signaling Technology, 9216B) |
| | MAP2K7 |
| | Anti-phospho MBP antibody (Upstate #05-429) |
| Detection reagent | MAP2K3 |
| | AlphaScreen GST Detection Kit |
| | (PerkinElmer #6760603M) |
| | MAP2K7 |
| | AlphaScreen IgG Detection Kit (Protein A) |
| | (PerkinElmer #6760617C) |
| Plate reader | Fusion α (PerkinElmer) |

5. Assail Procedures

ELISA

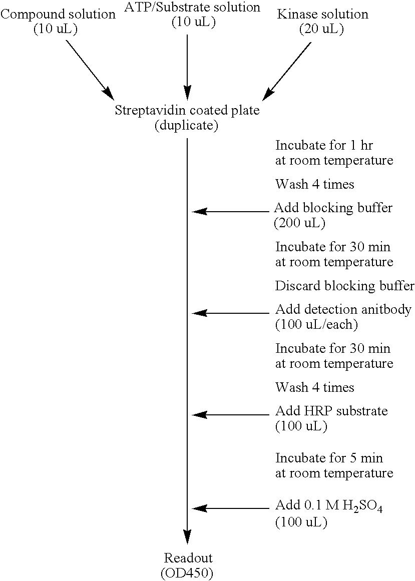

IMAP Assay

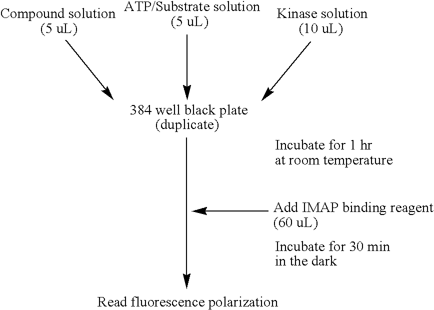

AlphaScreen Assay

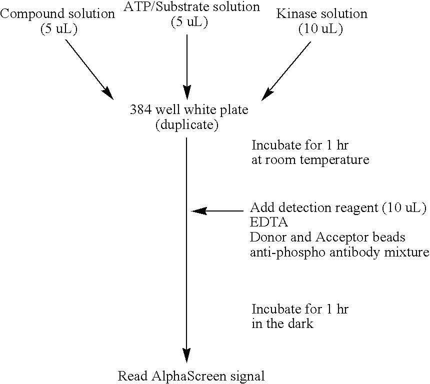

6. Assay Conditions

| | | Substrate* | | ATP (μM) | | Metal | |
|---|---|---|---|---|---|---|---|
| Kinase | Platform | Name | (nM) | Km | Assay | Name | (mM) |
| ABL | ELISA | Gast | 250 | 1.2 | 1 | Mg | 5 |
| ARG | ELISA | Gast | 250 | 6.7 | 5 | Mg | 10 |
| TNK1 | ELISA | Lyn | 250 | 7.1 | 10 | Mg | 40 |
| ALK | ELISA | Gast | 250 | 11 | 10 | Mg | 0.25 |
| AXL | ELISA | Lyn | 250 | 10 | 10 | Mn | 10 |
| MER | ELISA | Gast | 250 | 36 | 30 | Mg | 10 |
| CSK | ELISA | Gast | 250 | 10 | 10 | Mg | 10 |
| EGFR | ELISA | Lyn | 250 | 5.2 | 5 | Mg | 5 |
| HER2 | ELISA | Lyn | 250 | 5.9 | 5 | Mn | 10 |
| HER4 | ELISA | Lyn | 250 | 9.3 | 10 | Mg | 40 |
| EphA2 | ELISA | Lyn | 250 | 0.54 | 0.5 | Mn | 10 |
| EphB2 | ELISA | Lyn | 250 | 95 | 100 | Mg | 20 |
| EphB4 | ELISA | Lyn | 250 | 200 | 100 | Mg | 20 |
| FAK | ELISA | Gast | 250 | 14 | 10 | Mg | 5 |
| FGFR1 | ELISA | Lyn | 250 | 54 | 50 | Mg | 20 |
| FGFR2 | ELISA | Gast | 250 | 20 | 20 | Mg | 20 |
| IGF1R | ELISA | Gast | 250 | 180 | 100 | Mg | 10 |
| INSR | ELISA | Gast | 250 | 64 | 60 | Mg | 10 |
| JAK1 | ELISA | IRS1 | 250 | 7.9 | 10 | Mg | 5 |
| JAK2 | ELISA | Lyn | 250 | 7.4 | 5 | Mg | 20 |
| JAK3 | ELISA | Lyn | 250 | 3.6 | 5 | Mg | 10 |
| TYK2 | ELISA | IRS1 | 250 | 6.2 | 10 | Mg | 10 |
| MET | ELISA | IRS1 | 250 | 18 | 20 | Mg | 5 |
| RON | ELISA | Lyn | 250 | 2 | 2 | Mn | 10 |
| FLT3 | ELISA | Gast | 250 | 79 | 100 | Mg | 20 |
| FMS(CSFR) | ELISA | Gast | 250 | 93 | 100 | Mg | 20 |
| KIT | ELISA | IRS1 | 250 | 35 | 30 | Mg | 2.5 |
| PDGFRα | ELISA | IRS1 | 250 | 26 | 25 | Mg | 5 |
| PDGFRβ | ELISA | IRS1 | 250 | 15 | 10 | Mg | 5 |
| RET | ELISA | Gast | 250 | 7 | 5 | Mg | 20 |
| BLK | ELISA | Gast | 250 | 11 | 10 | Mg | 20 |
| BRK | ELISA | Gast | 250 | 16 | 20 | Mn | 20 |
| FGR | ELISA | Gast | 250 | 4.6 | 5 | Mg | 20 |
| FYN | ELISA | Gast | 250 | 20 | 20 | Mg | 20 |
| HCK | ELISA | Gast | 250 | 22 | 20 | Mg | 10 |
| LCK | ELISA | Gast | 250 | 64 | 50 | Mg | 20 |
| LYNa | ELISA | Gast | 250 | 28 | 30 | Mg | 10 |
| LYNb | ELISA | Gast | 250 | 61 | 60 | Mg | 10 |
| SRC | ELISA | Lyn | 250 | 28 | 25 | Mg | 5 |
| SRM | ELISA | Gast | 250 | 7.8 | 10 | Mn | 10 |
| YES | ELISA | Gast | 250 | 38 | 40 | Mg | 10 |
| TRKA | ELISA | Lyn | 250 | 110 | 100 | Mg | 20 |
| TRKB | ELISA | IRS1 | 250 | 120 | 100 | Mg | 10 |
| TRKC | ELISA | Lyn | 250 | 230 | 100 | Mg | 0.5 |
| FLT1 | ELISA | Lyn | 250 | 23 | 20 | Mg | 20 |
| KDR | ELISA | Lyn | 250 | 120 | 100 | Mg | 40 |
| PKACα | IMAP | GS | 250 | 1.2 | 1 | Mg | 5 |
| PKCα | IMAP | PKCq | 250 | 8 | 10 | Mg | 1 |
| PKCε | IMAP | PKCq | 25 | 4 | 4 | Mg | 2.5 |
| PKCγ | IMAP | PKCq | 25 | 2.5 | 2 | Mg | 2.5 |
| CaMK4 | IMAP | Synapsin | 100 | 46 | 50 | Mg | 20 |
| CaMK2α | IMAP | CaMK2a | 100 | 21 | 20 | Mg | 5 |
| CHK1 | IMAP | CHK1tide | 250 | 6.5 | 10 | Mg | 5 |
| MAPKAPK2 | IMAP | GS | 100 | 12 | 10 | Mg | 1 |
| CHK2 | IMAP | CHK1tide | 250 | 9 | 10 | Mg | 5 |
| CDK2/cyclinA | IMAP | Histon H1 | 100 | 25 | 30 | Mg | 5 |
| Erk1 | IMAP | Erktide | 100 | 10 | 10 | Mg | 10 |
| Erk2 | IMAP | Erktide | 100 | 8.6 | 10 | Mg | 10 |
| JNK1 | ELISA | ATF2 | 50 | 12 | 10 | Mg | 10 |
| JNK2 | ELISA | ATF2 | 50 | 4.5 | 5 | Mg | 10 |
| p38α | IMAP | Erktide | 100 | 15 | 15 | Mg | 10 |
| p38β | IMAP | Erktide | 100 | 13 | 10 | Mg | 10 |
| AurA | IMAP | PKAtide | 100 | 28 | 30 | Mg | 5 |
| IKKβ | IMAP | IkBa | 100 | 14 | 20 | Mg | 10 |
| MAP2K3 | Alpha | p38α | 25 | 2.2 | 2 | Mg | 2 |
| MAP2K7 | Alpha | JNK1 | 50 | 17 | 25 | Mg | 5 |
| | (cascade) | MBP | 50 | | | | |
| IRAK4 | IMAP | SRPKtide | 100 | 79 | 80 | Mn | 2.5 |

7. Substrate Information

| Name | Description |
|---|---|
| Gast | Biotin-EGPWLEEEEEAYGWMDF-NH₂ |
| Lyn | Biotin-X-EQEDEPEGDYFEWLEPE |
| IRS1 | Biotin-X-KKSRGDYMTMQIG |
| GS | FITC-X-KKLNRTLSVA |
| PKCq | LHQRRGSIKQAKVHHKV(FITC)-NH₂ |
| Synapsin | FITC-X-LRRRLSDANF-NH₂ |
| CaMK2a | FITC-X-MHRQETVD-H₂ |
| CHK1tide | FITC-X-ALKLVRYPSFVITAK-NH₂ |
| Histon H1 | AAKAKKTPKKAKK(FITC)-NH₂ |
| Erktide | FITC-X-IPTTPITTTYFFFK-NH₂ |
| ATF2 | Biotinylated N-terminal GST-tagged recombinant protein |
| | containing amino acids 19-96 of human ATF2, expressed in |
| | E. coli |
| PKAtide | 5FAM-GRTGTTNSI-NH₂ |
| IkBa | 5FAM-GRHDSGDSMK-NH₂ |
| p38α | N-terminal GST-tagged recombinant protein expressed in |
| | E. coli |
| JNK1 | N-terminal His tagged recombinant protein expressed in |
| | E. coli |
| MBP | De-phosphorylated myelin basic protein conjugated with |
| | biotin |
| SRPKtide | FITC-X-RSRSRSRSRSRSRSR-NH₂ |

X = epsilon aminocaproic acid

8. Data Analysis

Readout value of reaction control (with ATP) was set as a 0% inhibition, and readout value of background (without ATP) was set as a 100% inhibition, and the percent inhibition of each test solution was calculated.

5.2 Results

TABLE 1
Tyrosine kinases

| | K-252a (200 nM) | Ref. | |
|---|---|---|---|
| Kinase | % Inhibition | % Inhibition | Ref. compound (nM) |
| ABL | −14.2 | 86.2 | Staurosporine (1000) |
| ARG | 0.1 | 89.6 | Staurosporine (3000) |
| TNK1 | 94.8 | 89.2 | Staurosporine (3) |
| ALK | 69.5 | 93.8 | Staurosporine (30) |
| AXL | 51.5 | 85.1 | Staurosporine (3000) |
| MER | 80.8 | 90.7 | Staurosporine (100) |
| CSK | 43.0 | 88.8 | Staurosporine (300) |
| EGFR | 16.4 | 91.4 | Staurosporine (10000) |
| HER2 | 7.7 | 65.6 | Staurosporine (10000) |
| HER4 | 39.4 | 94.1 | Staurosporine (10000) |
| EphA2 | 8.9 | 93.7 | Staurosporine (10000) |
| EphB2 | 0.4 | 83.7 | Staurosporine (1000) |
| EphB4 | 13.3 | 92.7 | Staurosporine (10000) |
| FAK | 53.5 | 90.4 | Staurosporine (100) |
| FGFR1 | 57.9 | 91.4 | Staurosporine (100) |
| FGFR2 | 67.2 | 96.7 | Staurosporine (100) |
| IGF1R | 40.6 | 98.6 | Staurosporine (3000) |
| INSR | 38.0 | 93.3 | Staurosporine (1000) |
| JAK1 | 83.1 | 82.8 | Staurosporine (30) |
| JAK2 | 99.5 | 96.3 | Staurosporine (3) |
| JAK3 | 100.4 | 97.8 | Staurosporine (3) |
| TYK2 | 98.1 | 94.5 | Staurosporine (3) |
| MET | 83.2 | 87.0 | Staurosporine (300) |
| RON | 5.2 | 81.6 | Staurosporine (10000) |
| FLT3 | 95.8 | 94.0 | Staurosporine (3) |
| CSFR | 55.7 | 89.1 | Staurosporine (30) |
| KIT | 87.3 | 96.7 | Staurosporine (10) |
| PDGFRα | 96.1 | 93.5 | Staurosporine (3) |
| PDGFRβ | 98.1 | 98.7 | Staurosporine (0.3) |
| RET | 94.2 | 88.1 | Staurosporine (30) |
| BLK | 83.5 | 94.9 | Staurosporine (30) |
| BRK | 35.8 | 87.4 | Staurosporine (10000) |
| FGR | 85.6 | 88.7 | Staurosporine (10) |
| FYN | 67.2 | 98.9 | Staurosporine (100) |
| HCK | 68.2 | 96.1 | Staurosporine (30) |
| LCK | 65.2 | 97.6 | Staurosporine (100) |
| LYNa | 58.9 | 95.2 | Staurosporine (100) |
| LYNb | 51.2 | 96.4 | Staurosporine (100) |
| SRC | 20.6 | 90.0 | Staurosporine (3000) |
| SRM | 0.4 | 74.0 | Staurosporine (10000) |
| YES | 65.1 | 94.1 | Staurosporine (30) |
| TrkA | 97.6 | 93.4 | Staurosporine (3) |
| TrkB | 98.2 | 67.3 | Staurosporine (0.3) |
| TrkC | 94.4 | 86.2 | Staurosporine (10) |
| FLT1 | 73.9 | 94.8 | Staurosporine (300) |
| KDR | 62.9 | 87.7 | Staurosporine (300) |

TABLE 2
Serine/Threonine kinases

| | | Ref. | |
|---|---|---|---|
| | K-252a (200 nM) | % | |
| Kinase | % Inhibition | Inhibition | Ref. cpd (nM) |
| PKACα | 69.4 | 94.9 | Staurosporine (30) |
| PKCα | 64.4 | 97.8 | Staurosporine (30) |
| PKCε | 60.9 | 98.8 | Staurosporine (3) |
| PKCγ | 53.3 | 94.3 | Staurosporine (10) |
| CaMK4 | 0.0 | 88.4 | Staurosporine (3000) |
| CaMK2α | 83.9 | 92.9 | Staurosporine (30) |
| CHK1 | 92.8 | 91.1 | Staurosporine (10) |
| MAPKAPK2 | 44.3 | 90.5 | Staurosporine (1000) |
| CHK2 | 93.9 | 97.1 | Staurosporine (300) |
| CDK/CycA | 75.5 | 95.7 | Staurosporine (30) |
| Erk1 | 32.0 | 80.2 | 5-Iodotubericidin (10000) |
| Erk2 | 14.2 | 62.1 | 5-Iodotubericidin (10000) |
| JNK1 | 94.3 | 82.1 | JNK Inhibitor II (300) |
| JNK2 | 91.4 | 92.8 | JNK Inhibitor II (300) |
| p38α | −1.5 | 87.9 | SB202190 (300) |
| p38β | −0.2 | 55.7 | SB202190 (300) |
| AurA | 91.9 | 84.9 | Staurosporine (30) |
| IKKβ | 2.7 | 47.9 | Staurosporine (10000) |
| MAP2K3 | 100.0 | 84.5 | Staurosporine (30) |
| MAP2K7 | 68.2 | 85.8 | Staurosporine (3000) |
| IRAK4 | 51.9 | 95.9 | Staurosporine (10000) |

### 6. Conclusions

The very potent inhibitory effects shown by K-252a in in vitro models of cell migration and proliferation makes K-252a a promising drug candidate to be used in the systemic or local therapy of HMGB1-related diseases.

Based on these results, it is clear that HMGB1, both released by injured/dead endothelial cells or secreted by activated circulating macrophages and monocytes, is one of the most relevant targets of restenosis after surgical angioplasty and of several severe pathologies in the field of inflammation and immunity. The inhibition of its role and activities of a typical chemotactic chemokine mainly in situ, exactly where the mechanical traumatic injury is caused and the process leading to restenosis formation begins and develops, seems to indicate preventive/therapeutic activities of a specific HMGB1 antagonist.

For the same reason, the systemic administration of inhibitors of the pathological activities induced by HMGB1 looks like a promising therapeutic approach to cure a wide panel of systemic and local illnesses. It is demonstrated here that K-252a is a potent in vitro inhibitor of the two activities of HMGB1 mainly involved in restenosis induction and formation as well as in the triggering, sustaining and amplifying of local and systemic inflammatory and immune responses.

In fact, at concentrations which are in the nanomolar range, it inhibited HMGB1-induced cell migration (FIG. 1) and proliferation (FIG. 2). This, translated in vivo, means that K-252a as a therapeutic agent alone or released by a stent embedded/coated with K252 itself in the lesioned site should actively antagonize HMGB1 by blocking/inhibiting smooth muscle cell shift from the phenotype characterized by absence of cell division and migration to the synthetic phenotype which leads them to run and proliferate into the injured blood vessel endothelial wall, producing restenosis. Similarly, in the case of therapy of inflammatory and immunological pathologies triggered and sustained by HMGB1, the systemic administration of K-252a should inhibit/block the pathological cascade induced by this chemotactic chemokine, with beneficial effects on the onset and the course of the illness.

Moreover, the in vivo data obtained for the treatment with K-252a of mice affected by severe endotoxemia induced by LPS with K-252a, further support the results achieved with the above described in vitro data of cell migration and proliferation. In fact, the administration of 6.7 mg/kg i.p. of K-252a, for 4 times to mice bearing a severe LPS-induced endotoxemia, increase significantly (over 60%) the survival of the tested mice compared to the survival of the control mice. The control mice in fact do not survive over the fifth day. The in vivo results thus show a remarkable decrease in mortality in mice treated with K-252a, confirming that K-252a is a promising drug candidate to be used in the systemic or local treatment of HMGB1-related pathologies.

The mechanism by which K-252a inhibits the activity of HMGB1 seems to be kinase inhibition. Therefore, other kinase inhibitors, in particular known kinase inhibitors, e.g. of tyrosine kinase, such as TrkA, TrkB, TrkC, of phosphokinase, such as PKA, PKC, PKG, or/and of a further kinase, such as Raf kinase, Ras kinase, CaM kinase, MLC kinase, MAP kinase, MEK, ERK, JUN kinase, PI3Kγ, may also be suitable compounds for inhibiting the activity of HMGB1 in vitro and in vivo. Therefore, such kinase inhibitors may provide a promising therapeutic approach in diseases involving activity of released HMBG1.

### REFERENCES

1. Palumbo R., Sampaolesi M., De Marchis F., Tonlorenzi R., Colombetti S., Mondino A., Cossu G., Bianchi M. E. (2004) Extracellular HMGB1, a signal of tissue damage, induces mesoangioblast migration and proliferation. The Journal of Cell Biology, 164: 441-449.
2. Scaffidi P., Misteli T., Bianchi M. E. (2002) Release of chromatin protein HMGB1 by necrotic cells triggers inflammation, Nature. 418: 191-195.
3. Andersson, U., Erlandsson-Harris, H., Yang, H. and Tracey, K. J. (2002) HMGB1 as a DNA-binding cytokine. J. Leucocyte Biol., 72: 1084-1091
4. Agresti, A. and Bianchi, M. E. (2003) HMGB-proteins and gene expression Current Opin. In Genetics and Develop., 13: 170-178
5. Degryse, B., de Virgilio, M. (2003) The nuclear protein HMGB1, a new kind of chemokine ? FEBS Letters, 553: 11-17
6. Kase H., Iwahashi K., Matsuda Y. (1986) K-252a, a potent inhibitor of protein kinase C from microbial origin J. Antibiot. (Tokyo). 39: 1059-1065.
7. Nakanishi S., Yamada K., Kase H., Nakamura S., Nonomura Y. (1988) K-252a, a novel microbial product, inhibits smooth muscle myosin light chain kinase, J. Biol. Chem. 263: 6215-6219.
8. Delsite R. and Djakiew D. (1996) Antiproliferative effect of the kinase inhibitor K-252a on human prostatic carcinoma cell lines, J. Androl. 17: 481-490.
9. Donovan M J., Miranda R. C., Kraemer R., McCaffrey T. A., Tessarolo L., Mahadeo D., Sharif S., Kaplan D. R., Tsoulfas P., Parada L., et al. (1995) Neurotrophin and neurotrophin receptors in vascular smooth muscle cells. Regulation of expression in response to injury, Am. J. of Pathology. 147(2): 309-324.
10. Schmidt, A. M., Yan, S. F. and Stem, D. M. (2001) The multigland receptor RAGE as a progression factor amplifying immune and inflammatory responses A. Clin. Invest, 108: 949-955.
11. Bonaldi, T., Talamo, F., Scaffidi, P., Ferrera, D., Porto, A., Bachi, A., Rubartelli, A., Agresti, A. and Bianchi M. E. (2003) Monocytic cells hyperacetylate chromatin protein HMGB1 to redirect it towards Secretion The EMBO Journal, 22: 5551-5560
12. Friedman, S. G., Czura, C1 J. and Tracey, K J. (2003) The gesture life of high mobility group box 1 Current Opinion in Clinical Nutrition and Metabolica Care, 6: 283-287
13. Gardella, S., Andrei, C, Ferrera, D., Lotti, L. V., Torrisi, M. R., Bianchi, M. E. And Rubartelli, A. (2002) The nuclear protein HMGB1 is secreted by monocytes via a non-classical, vesicle-mediated secretory pathway. EMBO. Rep., 3: 995-1001

1. Use of K-252a or/and a salt or a derivative thereof for the preparation of a medicament for the prevention or treatment of HMGB1-associated pathologies selected from the group consisting of HMGB1-induced or/and sustained restenosis, atherosclerosis and ischemia-reperfusion injury.

2. Use of claim 1, wherein the HMGB1-associated pathologies are pathologies associated with the non-acetylated form or/and the acetylated form of HMGB1 and pathologies associated with the nonacetylated form or/and the acetylated form of HMGB1 homologous proteins.

3. Use of claim 2, wherein the HMGB1 homologous proteins are HMGB2, HMGB3, HMG-1L10, HMG-4L or/and SP100-HMG.

4. The use of claim 1, wherein the HMGB1-associated pathologies are pathological conditions mediated by activation of the inflammatory cytokine cascade induced by HMGB1 or/and its homologous proteins.

5. The use of claim 1 for blocking, retarding or impairing connective tissue regeneration in restenosis during or after angioplasty.

6. The use of claim 1 in combination with a further agent.

7. The use of claim 6, wherein the further agent is capable of inhibiting early mediators of the inflammatory cytokine cascade.

8. The use of claim 7, wherein the further agent is an antagonist or inhibitor of a cytokine selected from the group consisting of TNF, IL-Ia, IL-13, ILR8i IL-8, MIP-Ia, MIP1β, MIP-2. MIF and IL-6.

9. The use of claim 6, wherein the further agent is an antibody to RAGE, a nucleic acid or nucleic acid analogue capable of inhibiting RAGE expression, e.g. an antisense molecule, a ribozyme or a RNA interference molecule, or a small synthetic molecule antagonist of the HMGB1 interaction with RAGE or soluble RAGE (sRAGE).

10. The use of claim 6, wherein the further agent is an antagonist or inhibitor of HMGB1 or of its homologous proteins.

11. The use of claim 10, wherein the antagonist or inhibitor is an antagonist or inhibitor of the non-acetylated or/and the acetylated form of HMGB1 or of its homologous proteins.

12. The use of claim 6, wherein the further agent is an inhibitor of the interaction of a Toll-like receptor (TLR), in particular of TLR2, TLR4, TLR7, TLR8 or/and TLR9, with HMGB1, preferably a monoclonal or polyclonal antibody, a nucleic acid or nucleic acid analogue capable of inhibiting TLR expression, e.g. an antisense molecule, a ribozyme or a RNA interference molecule, or a synthetic molecule having a size of less than 1000 Dalton.

13. The use of claim 12, wherein the further agent is a known inhibitor of a Toll-like receptor, in particular of TLR2, TLR4, TLR7, TLR8 or/and TLR9, in particular a nucleic acid or nucleic acid analogue capable of inhibiting TLR expression, e.g. an antisense molecule, a ribozyme or a RNA interference molecule.

14. The use of claim 6, wherein the further agent is the N-terminal lectine-like domain (D1) of thrombomodulin.

15. The use of claim 6, wherein the further agent is a synthetic double-stranded nucleic acid or nucleic acid analogue molecule with a bent shape structure.

16. The use of claim 15, wherein the synthetic double-stranded nucleic acid or nucleic acid analogue molecule is a double-stranded bent or cruciform DNA, PNA or DNA/PNA chimera or hybrid.

17. A pharmaceutical composition comprising an effective amount of K-252a or/and a salt or a derivative thereof as an active ingredient for the treatment of HMGB1-associated pathologies selected from the group consisting of HMGB1-induced or/and sustained restenosis, atherosclerosis and ischemia-reperfusion injury and pharmaceutically acceptable carriers, diluents and/or adjuvants.

18. The pharmaceutical composition of claim 17, wherein the HMGB1-associated pathologies are pathologies associated with the non-acetylated or/and the acetylated form of HMGB1 and pathologies associated with the non-acetylated or/and the acetylated form of HMGB1 homologous proteins.

19. The pharmaceutical composition of claim 18, wherein the HMGB1 homologous proteins are HMGB2, HMGB3, HMG-1L10, HMG-4L or/and SP100-HMG.

20. A pharmaceutical composition of claim 17, wherein the active ingredient is in combination with a further agent.

21. The use according to claim 1, wherein K-252a or/and a salt or a derivative thereof is reversibly immobilised on the surface of a medical device.

22. The use of claim 21, wherein K-252a or/and a salt or a derivative thereof is coated on or embedded in the surface of the medical device.

23. The use of claim 21, wherein the medical device is selected from surgical instruments, implants, catheters or stents.

24. A medical device reversibly coated or/and embedded with K-252a or/and a salt or a derivative thereof.

25. The medical device of claim 24, wherein the medical device is selected from surgical instruments, implants, catheters or stents.
